(19) United States
(12) Patent Application Publication
(10) Pub. No.: US 2003/0165452 A1
(43) Pub. Date: Sep. 4, 2003

(54) Controlled released powder entrapment compositions for hydrophobic ingredient

(75) Inventors: Anthony D. Gonzalez, Waldwick, NJ (US); Glen T. Anderson, Cortland Manor, NY (US); John A. Duffy, West Milford, NJ (US)

Correspondence Address: Charles N.J. Ruggiero, Esq., Ohlandt, Greeley, Ruggiero & Perle, L.L.P., 10th Floor, One Landmark Square, Stamford, CT, 06901-2682, US

(73) Assignee: AVON PRODUCTS, INC., NEW YORK, NY (US)

(21) Appl. No.: 10/032,801

(22) Filed: Dec. 27, 2001

(51) Int. Cl. A61K 7/06; A61K 7/11

(52) U.S. Cl. 424/70.12

(57) ABSTRACT

There are provided controlled release compositions and methods of making thereof. The compositions have an effective amount of at least one hydrophobic active ingredient and a silicone fluid entrapped in a hybrid silicone powder matrix. The present compositions preferably are dry powders.

### BACKGROUND OF THE INVENTION

1. Field of the Invention

The present invention relates to time or controlled release topical compositions. More particularly, the present invention relates to compositions and methods for delivering hydrophobic, liquid active ingredients to skin in powder form. The compositions of the present invention have one or more hydrophobic active ingredients and a volatile silicone fluid entrapped in a hybrid silicone powder matrix.

2. Description of the Prior Art

As used herein, the term “hydrophobic” means oil soluble and/or oil dispersible. There are a variety of hydrophobic active ingredients used in the personal care and cosmetic industries, including but not limited to, skin care ingredients and many insect repellents.

The use of insect repellents is one approach to reduce the spread of insect-borne diseases. However, there is general concern about over exposure to insect repellents by consumers, since the effects of such exposure may be harmful at certain concentrations. Thus, insect repellents would ideally be effective at the lowest possible concentration, thus maximizing their efficiency and minimizing the user's exposure to the repellent. Accordingly, it is desired to have insect repellent compositions that provide slow, controlled or time-release properties. This provides for a reduced amount of repellent in use and for extended periods of time.

Encapsulation technology is widely used in a number of industries for controlled release purposes. For example, the pharmaceutical industry employs encapsulation as a way of providing effective drug delivery. However, the manufacturing of encapsulation products and release mechanisms are often complicated. Release mechanisms may, for example, include shearing, specific pH requirements, specific chemical or ionic conditions, specific moisture levels, or particular temperature levels, and the like.

U.S. Pat. No. 6,106,849 to Malkan et al. is directed to a water soluble dry foam personal care product. This patent provides a specific insect repellent, a pyrethrum derivative, in a silicone elastomer matrix. The silicone elastomer is synthesized in the presence of the repellent, and the repellent is then chemically reacted with the base material to form the elastomer matrix.

U.S. Pat. No. 6,183,765 B1 to Kalder et al. is directed to silicone elastomers with insecticidal effect. This patent provides a pyrethroid insecticide that is incorporated in a silicone elastomer at the time of synthesis.

A large number of compositions have been developed for human use that provide insect repellent and skin care properties. These compositions may also-have ingredients for anti-aging, skin moisturization, and UV protection, among others. Most active ingredients in such compositions are hydrophobic, and are commonly dispersed in an anhydrous system or in the oil phase of an emulsion. Providing hydrophobic active ingredients, that are soluble and/or dispersible in oil or other non-polar fluid materials, in a powder form suitable for consumer use is a technical challenge. Development of a convenient powder-based system for topical delivery of hydrophobic active ingredients in a time-controlled fashion is highly desirable.

In particular, a topical, powdered insect repellent composition, having one or more hydrophobic active ingredients, that is effective, easily manufactured, and useful for topical applications and for fabric impregnation in clothing, sporting goods, athletic equipment and the like, would be highly desired.

### SUMMARY OF THE INVENTION

It is an object of the present invention to provide time or controlled-release powder compositions with one or more hydrophobic active ingredients.

It is another object of the present invention to provide powdered compositions that entrap one or more hydrophobic active ingredients.

It is yet another object of the present invention to provide insect repellent compositions having an insect repellent active with time or controlled release capability.

It is a further object of the present invention to provide compositions having hydrophobic active ingredient that have time or controlled release activity in which the compositions are in a cream, gel, liquid, lotion, mousse, patch, powder, stick or spray product form. It is a still further object of the present invention to provide insect repellent compositions having time or controlled release insect repellent activity in which the composition is a powder.

These and other objects and advantages of the present invention and equivalents thereof, are achieved by compositions that have a controlled release hydrophobic active ingredient. The compositions have an effective amount of at least one hydrophobic active ingredient entrapped in a hybrid silicone powder matrix.

Also, the present invention provides a method for the production of controlled release compositions having one or more hydrophobic active ingredients.

Further, the present invention provides a method for treating skin and/or repelling insects by the use of the controlled release compositions of the present invention.

### DETAILED DESCRIPTION OF THE INVENTION

The present invention provides timed or controlled release compositions having a hydrophobic active ingredient or combination of hydrophobic ingredients that are entrapped in a hybrid silicone powder matrix. In a preferred embodiment, one or more hydrophobic insect repellents are entrapped in an organomodified dimethicone crosspolymer matrix to provide time or controlled release of the insect repellent. The present invention also provides a process for making a hybrid silicone powder matrix, and for entrapping hydrophobic active ingredients in a powder form.

When used in the present invention, entrap means entangled or adsorbed within a polymer or polymer matrix in a fashion analogous to the retention of water in a sponge. It does not mean encapsulated.

As will be described in more detail herein with respect to the process of the present invention, the present hybrid silicone powder matrix is a combination of a hybrid silicone powder and a volatile silicone.

The hybrid silicone powder of the present invention is a cross between a silicone rubber powder and a silicone resin powder.

Silicone rubber powder and silicone resin powder are individually known in the art and available for use with cosmetic and personal care products. Silicone rubber powder, which has excellent smoothness and softness, finds use in a variety of products to improve the feel upon application to the skin. However, a distinct disadvantage of the silicone rubber powder is that is has a strong tendency to aggregate. This causes products having this powder to exhibit inferior or poor dispersion. In contrast, silicone resin powder does not have such a tendency to aggregate. However, silicone resin powder has poor smoothness and softness qualities resulting in its limited use in cosmetic products.

The hybrid silicone powder of the present invention overcomes the deficiencies of using silicone rubber powder or silicone resin powder individually. The hybrid silicone powder of the present invention was developed by coating the surface of silicone rubber powder particles with silicone resin. The resin coating chemically binds to the rubber components and, thus, prevents the release or separation of the coating from the rubber. As a result, the softness of the silicone rubber powder is preserved while the tendency of the silicone rubber powders to aggregate is alleviated. Thus, the hybrid silicone powder of the present invention combines the advantages of the soft feel of the silicone rubber powder with the slip and lubricity of the silicone resin powder, without the disadvantages of either. The silicone rubber powder is preferably comprised of spherical particles. The hybrid silicone powder is a crosspolymer selected from, but not limited to, silicone, dimethicone, cyclomethicone, vinyl dimethicone, organomodified silicone, organomodified dimethicone, and any combinations thereof. A preferred hybrid silicone powder is vinyl dimethicone/methicone silsesquioxane crosspolymer, available commercially as KSP 101 from Shin-Etsu Silicones. In the present invention, the hybrid silicone powder is present in the compositions in an amount about 0.1 wt % to about 99 wt %, and preferably about 5 wt % to about 80 wt %, of the total weight of the composition.

The volatile silicone of the present invention may conveniently be selected from, but is not limited to, the group of linear and cyclic silicones having 3 to 8 silicone molecules. Preferred volatile silicone(s) are cyclic silicones having 4 to 6 silicone molecules. Most preferred is cyclomethicone. The volatile silicone is present in the composition of the present invention in an amount about 1 wt % to about 99 wt % and preferably about 5 wt % to about 80 wt %, of the total weight of the composition. The addition of a volatile silicone fluid to the hybrid silicone powder causes the hybrid silicone powder to swell into a crosslinked hybrid silicone powder matrix that entraps any hydrophobic active(s) added to the hybrid silicone powder.

The present invention is useful to entrap any hydrophobic active ingredient, or combination of hydrophobic ingredients, that is in liquid form or, if a solid, may be pre-dissolved in a suitable essentially hydrophobic liquid (i.e., oil) before addition to the hybrid silicone powder. One or more hydrophobic active ingredients is selected from, but are not limited to, an insect repellent, sunscreen, retinol, retinoic acid, retinyl palmitate, oil soluble retinol derivative, ascorbyl palmitate, oil soluble ascorbic acid derivative, salicylic acid, derivative of salicylic acid, fragrance, phytol, essential oil, perilla oil, tamarind oil, or any combinations thereof. One of the active ingredients is preferably an insect repellent. In each insect repellent composition of the present invention, at least one predominantly hydrophobic insect repellent active is selected. However, it should be understood that mixtures of insect repellent actives, including hydrophobic and hydrophilic insect repellent actives, may be used in the present compositions.

The one or more insect repellent actives used in the present compositions include, but are not limited to: oil of citronella, N,N diethyl-m-toluamide (DEET), ethyl butylacetylaminopropionate (IR 3535; Merck Co.), hydroxy-ethyl isobutyl piperidine carboxylate (1-piperidine carboxylic acid; Bayer KBR 3023), dimethyl phthalate, 2-ethyl-1,3 hexanediol, soybean oil, neem oil, lemon grass oil, geranium/geraniol oil, p-methane-3,8-diol, other botanical extracts having insect repellent properties, or any combinations thereof. Other insect repellent actives that can be used in the present invention are disclosed in U.S. Pat. Nos. 5,130,136 and 5,698,209, which are incorporated herein by reference. The preferred insect repellents for use in the compositions of the present invention are N,N diethyl-m-toluamide, ethyl butylacetylaminopropionate, oil of citronella, hydroxyethyl isobutyl piperidine carboxylate, p-menthane-3,8-diol, or any combinations thereof.

The one or more hydrophobic actives in the compositions of the present invention may be present in an amount about 0.01 wt % to about 90 wt % based on the total weight of the composition. The one or more hydrophobic actives preferably are present in an amount about 0.05 wt % to about 60 wt %, and more preferably about 0.05 wt % to about. 45 wt %, based on the total weight of the composition.

The amount of the hybrid silicone powder matrix in the compositions of the present invention is about 0.1 wt % to about 99 wt %, based on the total weight of the composition. Preferably, the amount of the matrix is about 10 wt % to about 50 wt %, based on the total weight of the composition.

A preferred hybrid silicone powder matrix with one or more entrapped insect repellent actives or other hydrophobic active(s) is made as follows. The hydrophobic actives are thoroughly mixed with the hybrid silicone powder. This mixing allows the organic groups of the hybrid silicone powder to associate fully with the active(s). The mixing is done for a period of time and at a temperature sufficient to form a uniform blend. When the mixture is completely uniform, a viscous (i.e., gel or gel-like dispersion/slurry) blend is formed. A volatile silicone fluid is added and mixed with the gel or slurry blend in an effective amount and for a period of time and at a temperature sufficient to cause the blend to “swell”, forming a matrix that entraps both the fluid and the active. The swelling occurs because of the similarity in structure between the silicone fluid and the silicone powder. Room temperature is preferred. An indication that the matrix/entrapment system is being formed is shown by a change in the form of the blend from a viscous slurry or gel to a light, dry powder.

When this powder is applied and rubbed into skin, the silicone fluid and the active are both released. This causes the powder to transform into a smooth, silky and transparent film on the skin. The hybrid nature of the powder combines the soft feel of silicone rubber with the slip and lubricity of silicone resins, adding to the favorable aesthetic attributes.

The hybrid silicone powder matrix can be custom formulated to modify the rate of release of the hydrophobic active(s). For example, the volatility of the silicone fluid or the level of cross-polymerization of the hybrid silicone powder may be varied to modify the rate of release of active. The resultant compositions provide time/controlled release of the one or more entrapped actives from the matrix.

The compositions of the present invention can be placed in a cosmetically acceptable vehicle. The matrix can be placed in the vehicle after entrapment of the active in the matrix. Such vehicles include one or more ingredients selected from the group of aqueous systems, glycerin, C₁₋₄ alcohols, fatty alcohols, fatty ethers, fatty esters, polyols, glycols, vegetable oils, mineral oils, liposomes, laminar lipid materials, silicone oils, water, and any combinations thereof.

The final compositions of the invention may additionally contain free or non-entrapped insect repellent or other active(s), or blends of actives, to further enhance the immediate efficacy of the compositions. Such additional additive(s) may add stability to the compositions, enhance the activity of other actives, and produce a synergistic effect.

Optionally, other materials, such as one or more vitamins and antioxidants, may be added to compositions of the invention. Such vitamins and antioxidants include, but are not limited to, vitamins E, F and K, and polyphenols.

Preferably, the resultant controlled release compositions of the present invention are dry, powdered forms. These compositions can have relatively high levels of liquid insect repellent or other active. Such powdered compositions may be applied topically, added to other materials, and/or impregnated into fabrics, clothing, plastics, table cloths, bracelets, laundry sheets, satchels, candles, coils, soaps, and the like. The inherent water resistance of the silicone matrix imparts superior water resistance to the one or more insect repellent or other actives incorporated into the matrix.

Also, the powder compositions of the present invention having time or controlled release insect repellent or other activity can alone or together with the vehicle be modified to be in the form of a aerosol spray, cream, emulsion, solid, liquid, dispersion, foam, gel, lotion, mousse, ointment, powder, patch, pomade, solution, pump spray, stick, and towelette.

The present invention provides a novel way to deliver liquid hydrophobic actives to the skin in a powdered form. The key is the use of the hybrid silicone rubber/resin powder as the entrapment agent. Although silicone resins and silicone rubber powders have previously been dispersed individually in various types of oils, the resulting product is an oil or gel itself, not a powder. The present inventors have surprisingly discovered that a combined hybrid silicone rubber/resin powder material allows entrapment of liquid, hydrophobic actives in a unique powdered form. Because the actives are entrapped rather than encapsulated, the complete amount of active is available for controlled delivery without need for a specific release mechanism such as temperature or moisture. In addition to the performance benefits, this invention allows for very high levels of such hydrophobic actives to be incorporated into an elegant time-released powder.

Although the present invention describes in detail certain embodiments, it is understood that variations and modifications exist known to those skilled in the art that are within the invention. Accordingly, the present invention is intended to encompass all such alternatives, modifications and variations that are within the scope of the invention as set forth in the following claims.

What is claimed is:

1. A topical composition comprising:
a hybrid silicone powder matrix; and
an effective amount of a hydrophobic active ingredient and a volatile silicone entrapped within said hybrid silicone powder matrix.

2. The composition of claim 1, wherein the composition is a time/controlled release composition.

3. The composition of claim 1, wherein said hybrid silicone powder matrix comprises a hybrid silicone powder.

4. The composition of claim 3, wherein said hybrid silicone powder is silicone rubber powder particles coated with silicone resin powder.

5. The composition of claim 3, wherein said hybrid silicone powder is a crosspolymer selected from the group consisting of silicone, dimethicone, cyclomethicone, vinyl dimethicone, organomodified silicone, organomodified dimethicone, and any combinations thereof.

6. The composition of claim 3, wherein said hybrid silicone powder is a vinyl dimethicone/methicone silsesquioxane crosspolymer.

7. The composition of claim 3, wherein said hybrid silicone powder is present in an amount about 0.1 wt % to about 99% of the total weight of the composition.

8. The composition of claim 3, wherein said hybrid silicone powder is present in an amount from about 5 wt % to about 80% of the total weight of the composition.

9. The composition of claim 1, wherein said volatile silicone is selected from the group consisting of linear and cyclic silicones having 3 to 8 silicone molecules.

10. The composition of claim 9, wherein said volatile silicone is a cyclomethicone.

11. The composition of claim 1, wherein said hydrophobic active ingredient is selected from the group consisting of an insect repellent, sunscreen, retinol, retinoic acid, retinyl palmitate, oil soluble retinol derivative, ascorbyl palmitate, oil soluble ascorbic acid derivative, salicylic acid, derivative of salicylic acid, fragrance, phytol, essential oil, perilla oil, tamarind oil, and any combinations thereof.

12. The composition of claim 1, wherein said hydrophobic active ingredient is present in an amount about 0.01 wt % to about 90% of the total weight of the composition.

13. The composition of claim 1, wherein said hydrophobic active ingredient is present in an amount about 0.05 wt % to about 60 wt % of the total weight of the composition.

14. The composition of claim 1, wherein said hydrophobic active ingredient is present in an amount about 0.05 wt % to about 45 wt % of the total weight of the composition

15. The composition of claim 1, wherein said hybrid silicone powder matrix is present in an amount about 0.1 wt % to about 99 wt % of the total weight of the composition.

16. The composition of claim 1, wherein said hybrid silicone powder matrix is present in an amount about 10 wt % to about 50 wt % of the total weight of the composition.

17. The composition of claim 1, wherein said volatile silicone is present in an amount about 1 wt % to about 99 wt % of the total weight of the composition.

18. The composition of claim 1, wherein said volatile silicone is present in an amount about 5 wt % to about 80 wt % of the total weight of the composition.

19. The composition of claim 1, wherein said hydrophobic active ingredient is one or more insect repellent actives selected from the group consisting of oil of citronella, N,N diethyl-m-toluamide, ethyl butylacetylaminopropionate, hydroxy-ethyl isobutyl piperidine carboxylate, dimethyl phthalate, 2-ethyl-1,3 hexanediol, neem oil, soybean oil, lemon grass oil, geranium/geraniol oil, p-methane-3,8-diol, and any combinations thereof.

20. The composition of claim 1, further comprising a cosmetically acceptable vehicle.

21. The composition of claim 1, wherein the composition is in a product form selected from the group of an aerosol spray, cream, emulsion, solid, liquid, dispersion, foam, gel, lotion, mousse, ointment, powder, patch, pomade, solution, pump spray, stick, and towelette.

22. A method for repelling insects from skin comprising topically applying to skin the composition of claim 1.

23. An insect repellent composition comprising:
a hybrid silicone powder matrix having a hybrid silicone crosspolymer powder selected from the group of dimethicone, cyclomethicone, vinyl dimethicone, organomodified variations thereof, and any combinations thereof; and
an effective amount of an insect repellent entrapped in said hybrid silicone powder matrix, wherein said insect repellent is one or more actives selected from the group consisting of oil of citronella, N,N diethyl-m-toluamide, ethyl butylacetylaminopropionate, hydroxy-ethyl isobutyl piperidine carboxylate, dimethyl phthalate, 2-ethyl-1,3 hexanediol, neem oil, soybean oil, lemon grass oil, geranium/geraniol oil, p-methane-3,8-diol, and any combinations thereof.

24. The composition of claim 23, wherein the composition is a time/controlled release composition.

25. A method for the production of a topical composition comprising:
mixing a hybrid silicone powder with at least one liquid hydrophobic ingredient for a period of time and at a temperature sufficient to form a gel or slurry; and
adding volatile silicone fluid to said gel or slurry; and thereafter
mixing said gel or slurry for a period of time and at a temperature sufficient to form a hybrid silicone powder matrix, wherein said hydrophobic ingredient and said silicone fluid are entrapped within said powder matrix.

26. The method of claim 25, wherein said hybrid silicone powder is a crosspolymer selected from the group consisting of silicone, dimethicone, cyclomethicone, vinyl dimethicone, organomodified silicone, organomodified dimethicone, and any combinations thereof.

27. The method of claim 25, wherein said hybrid silicone powder is a vinyl dimethicone/methicone silsesquioxane crosspolymer.

28. The method of claim 25, wherein said volatile silicone fluid is selected from the group consisting of linear and cyclic silicones having 3 to 8 silicone molecules.

29. The method of claim 25, wherein said hydrophobic active ingredient is selected from the group consisting of an insect repellent, sunscreen, retinol, retinoic acid, retinyl palmitate, oil soluble retinol derivative, ascorbyl palmitate, oil soluble ascorbic acid derivative, salicylic acid, derivative of salicylic acid, fragrance, phytol, essential oil, perilla oil, tamarind oil, and any combinations thereof.

30. The method of claim 25, wherein said hydrophobic active ingredient is present in an amount about 0.01 wt % to about 90% of the total weight of the composition.

31. The method of claim 25, wherein said hydrophobic active ingredient is present in an amount about 0.05 wt % to about 60 wt % of the total weight of the composition.

32. The method of claim 25, wherein said hydrophobic active ingredient is present in an amount about 0.05 wt % to about 45 wt % of the total weight of the composition.

33. The method of claim 25, wherein said hybrid silicone powder matrix is present in an amount about 0.1 wt % to about 99 wt % of the total weight of the composition.

34. The method of claim 25, wherein said hybrid silicone powder matrix is present in an amount about 10 wt % to about 50 wt % of the total weight of the composition.

35. The method of claim 25, wherein said volatile silicone fluid is present in an amount about 5 wt % to about 80 wt % of the total weight of the composition.

36. The method of claim 21, wherein said hydrophobic active ingredient is one or more insect repellent actives selected from the group consisting of oil of citronella, N,N diethyl-m-toluamide, ethyl butylacetylaminopropionate, hydroxy-ethyl isobutyl piperidine carboxylate, dimethyl phthalate, 2-ethyl-1,3 hexanediol, neem oil, soybean oil, lemon grass oil, geranium/geraniol oil, p-methane-3,8-diol, and any combinations thereof.

37. A hybrid silicone powder matrix comprising a hybrid silicone powder and a volatile silicone.

38. The matrix of claim 37, further comprising a cosmetically acceptable vehicle.

39. The matrix of claim 37, wherein said hybrid silicone powder comprises silicone rubber powder particles coated with silicone resin powder.

40. The matrix of claim 37, wherein said hybrid silicone powder is a crosspolymer selected from the group consisting of silicone, dimethicone, cyclomethicone, vinyl dimethicone, organomodified silicone, organomodified dimethicone, and any combinations thereof.

41. The matrix of claim 37, wherein said hybrid silicone powder is a vinyl dimethicone/methicone silsesquioxane crosspolymer.

42. The matrix of claim 37, wherein said volatile silicone is selected from the group consisting of linear and cyclic silicones having 3 to 8 silicone molecules.

43. The matrix of claim 42, wherein said volatile silicone is a cyclomethicone.

44. The matrix of claim 37, further comprising a hydrophobic active ingredient.

45. The matrix of claim 44, wherein said hydrophobic active ingredient is entrapped in the matrix.

46. The matrix of claim 44, wherein is selected from the group consisting of an insect repellent, sunscreen, retinol, retinoic acid, retinyl palmitate, oil soluble retinol derivative, ascorbyl palmitate, oil soluble ascorbic acid derivative, salicylic acid, derivative of salicylic acid, fragrance, phytol, essential oil, perilla oil, tamarind oil, and any combinations thereof.

47. The matrix of claim 44, wherein said hydrophobic active ingredient is one or more insect repellent actives selected from the group consisting of oil of citronella, N,N diethyl-m-toluamide, ethyl butylacetylaminopropionate, hydroxy-ethyl isobutyl piperidine carboxylate, dimethyl phthalate, 2-ethyl-1,3 hexanediol, neem oil, soybean oil, lemon grass oil, geranium/geraniol oil, p-methane-3,8-diol, and any combinations thereof.

48. A method of applying a liquid ingredient to skin in powder form comprising topically applying to skin the composition of claim 1, wherein the hydrophobic active ingredient and the volatile silicone are liquid.

49. A topical cosmetic composition comprising an effective amount of a hydrophobic active ingredient, an entrapment agent for the active ingredient, and a cosmetically acceptable vehicle, wherein the entrapment agent comprises a matrix of silicone rubber powder particles and silicone resin powder, and wherein the composition is in powder form upon topical application.
